(19) United States
(12) Patent Application Publication
(10) Pub. No.: US 2020/0338373 A1
(43) Pub. Date: Oct. 29, 2020

(54) OXYGEN TANK COVER

(71) Applicant: O2CUTIES LLC, Vassar, MI (US)

(72) Inventor: Susan TRINKLEIN, Vassar, MI (US)

(21) Appl. No.: 16/395,452

(22) Filed: Apr. 26, 2019

(51) Int. Cl. A62B 25/00; A61M 16/00; A62B 7/02

(52) U.S. Cl. CPC A62B 25/00; A62B 7/02; A61M 16/0003

(57) ABSTRACT

A cover and/or decorative cover for an oxygen tank. Covers that encompass or wrap an oxygen tank, oxygen cylinder and/or cart holding or supporting an oxygen tank and/or oxygen cylinder and associated valves, tubing and wiring.

### FIELD OF THE INVENTION

The present invention relates to a cover and/or decorative cover for an oxygen tank. More specifically, the present invention relates to covers that encompass or wrap an oxygen tank, oxygen cylinder and/or cart holding or supporting an oxygen tank and/or oxygen cylinder and associated valves, tubing and wiring.

### BACKGROUND OF THE INVENTION

Oxygen containers, tanks and/or or cylinders are used by patients to receive purified oxygen in instances where a patient is sick or unhealthy. Many medical patients are required to carry oxygen tanks as part of their daily lives and for their daily activities.

However, oxygen tanks are often cumbersome and unsightly and patients that are required to carry and transport an oxygen tank in their daily lives are often regarded as being sick and are avoided by the general public. This causes the mood of patients with oxygen tanks to sour and often patients that are required to carry oxygen tanks do not leave their homes and are not active in social events.

There are existing storage containers and holders for oxygen tanks. Existing systems include: U.S. Pat. No. 7,341,377 entitled CYLINDRICAL CONTAINER BAGS; U.S. Pat. No. 8,011,634 entitled OXYGEN TANK ACCESSORY APPARATUS; JP5838253B1 entitled EXTERNAL FUEL TANK COVER; U.S. Pat. No. 8,302,830 entitled SHOULDER HOLSTER FOR OXYGEN TANKS; US2013320054 entitled BAG; CN202961416 entitled OXYGEN HOSE PROTECTING BAG; and CN201643289 entitled CLOSE-FITTING OXYGEN BAG.

Disadvantageously however, none of these existing systems provide a cover for the oxygen tank that prevents the oxygen tank from being seen and viewed by others. The existing systems do not provide an effective way for medical patients to hide or cover an oxygen tank.

Attempting to overcome these disadvantages, it is desired to provide a method and system that solves the disadvantages in the prior art and to provide an oxygen tank cover that encompasses and/or wraps an oxygen tank, oxygen cylinder and/or cart holding or supporting an oxygen tank and/or oxygen cylinder and associated valves, tubing and wiring.

### SUMMARY OF THE INVENTION

It is an object of the invention positively affect the psychological impact of needing to use a portable oxygen system by making the clinical equipment appear whimsical by using a decorative cover.

It is an object of the present invention to provide a method and system of and for covering an oxygen tank, oxygen cylinder and/or cart holding or supporting an oxygen tank and/or oxygen cylinder and associated valves, tubing and wiring.

It is an object of the present invention to provide a method and system of and for covering an oxygen tank, oxygen cylinder and/or cart wherein the cover is a coat that wraps around that tank, wherein the coat has a “doll-like” configuration.

It is an object of the present invention to provide a method and system of and for covering an oxygen tank, oxygen cylinder and/or cart wherein the cover is easily removable.

It is an object of the present invention to provide a method and system of and for covering an oxygen tank, oxygen cylinder and/or cart wherein the cover is attached via Velcro® strips or spots so that it is easily removed as needed to replace the oxygen cylinder.

It is an object of the present invention to provide a method and system of and for covering an oxygen tank, oxygen cylinder and/or cart wherein the cover provides for support of the oxygen tank, so that the oxygen tank is protected from external elements.

These objects of the invention are achieved by providing a cover system for a pressurized medical gas cylinder, the cover system comprising: at least in part a flexible material; wherein the cover system configuration is easily removable from and/or added to the pressurized medical gas cylinder, wherein the cover system provides a less unsightly perception to and by a patient and others than that of the pressurized medical gas cylinder itself, and wherein the cover system facilitates the operation and replacement of the pressurized medical gas cylinder.

In certain embodiments, the cover further comprises at least one fastener. In certain embodiments, the at least one fastener is a Velcro® type fastener.

In certain embodiments, the pressurized medical gas cylinder includes an associated and accompanying holding and/or travel cart.

In certain embodiments, the pressurized medical gas cylinder includes associated and accompanying valves, tubing, dosing systems and masks, and wiring.

In certain embodiments, the cover wraps around the pressurized medical gas cylinder.

In certain embodiments, the cover is configured in appearance from the group consisting of toys, dolls, cartoon characters, action figures, animals, public figures, and combinations thereof.

In certain embodiments, the cover provides support for and to the pressurized medical gas cylinder.

In certain embodiments, the cover protects the pressurized medical gas cylinder from the group consisting of rain, water, heat, flame, snow, cold, high barometric pressure, low barometric pressure, and combinations thereof.

In certain embodiments, the cover comprises at least in part a natural material.

In certain embodiments, the cover comprises at least in part a synthetic material.

In certain embodiments, the cover is at least in part ridged.

In certain embodiments, the cover includes a face and eyes and includes various arms to cover the pressurized medical gas cylinder.

In certain embodiments, the cover includes at least two parts, a head portion and a body portion. In certain embodiments, the cover includes various accessories to accompany the cover to allow the “doll” to be dressed in clothing and other accessories.

In certain embodiments, the cover material has an opening at the rear to allow the cover material to slip onto and cover the pressurized medical gas cylinder.

In certain embodiments, the cover material includes an inner fastener, such that the inner fastener allows the cover material to be held to the pressurized medical gas cylinder.

In certain embodiments, the fabric material has holes and bores that allow for various wiring to extend therethrough.

In certain embodiments, the fabric material includes padding and other additional layers of material, so that the cover can have the proportions of a “doll.”

In certain embodiments, the fabric material includes multiple types of fabric, some of which offer structural support to the device.

Other objects of the invention are achieved by providing a method to cover a pressurized medical gas cylinder, the method comprising the steps of: providing a pressurized medical gas cylinder cover comprising at least in part a flexible material; wherein, the cover is configured to be easily removed from and/or added to the pressurized medical gas cylinder; wherein the cover system provides a less unsightly perception to and by a patient and others than that of the pressurized medical gas cylinder itself, and wherein the cover system facilitates the operation and replacement of the pressurized medical gas cylinder

In certain embodiments, the provided cover further comprises at least one fastener. In certain embodiments, the fastener is a Velcro® type fastener.

In certain embodiments, the pressurized medical gas cylinder includes an associated and accompanying holding and/or travel cart.

It In certain embodiments, the pressurized medical gas cylinder includes associated and accompanying valves, tubing, dosing systems and masks, and wiring.

In certain embodiments, the provided cover wraps around the pressurized medical gas cylinder.

In certain embodiments, the provided cover is configured in appearance from the group consisting of toys, dolls, cartoon characters, action figures, animals, public figures, and combinations thereof.

In certain embodiments, the provided cover provides support for and to the pressurized medical gas cylinder.

In certain embodiments, wherein the provided cover protects the pressurized medical gas cylinder from the group consisting of rain, water, heat, flame, snow, cold, high barometric pressure, low barometric pressure, and combinations thereof.

In certain embodiments, the provided cover comprises at least in part a natural material.

In certain embodiments, the provided cover comprises at least in part a synthetic material.

In certain embodiments, the provided cover is at least in part ridged.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is front perspective view schematic of an oxygen cover of an embodiment of the present invention of a woman with a dress;

FIG. 2 is a rear front perspective schematic of an oxygen cover of an embodiment of the present invention of a woman with a dress;

FIG. 3 is a side perspective schematic of an oxygen cover of an embodiment of the present invention of a woman with a dress;

FIG. 4 is a side perspective schematic of an oxygen cover of an embodiment of the present invention of a woman with a dress;

FIG. 5 is a top perspective schematic of an oxygen cover of an embodiment of the present invention of a woman with a dress.

### DETAILED DESCRIPTION OF THE INVENTION

In the following description, numerous details are set forth for the purpose of explanation. However, one of ordinary skill in the art will realize that the invention may be practiced without the use of these specific details.

The application incorporates the Appendix into the specification and the Appendix is part of the application as filed.

The purpose of this invention is to positively affect the psychological impact of needing to use a portable oxygen system by making the clinical equipment appear whimsical by using a decorative cover.

In certain embodiments, the product is a cloth “coat” that wraps around the tank and cart that has a doll-type head and arms attached. The doll head covers the cylinder controls which are easily accessed if needed. A dress is added over the doll form and can be changed seasonally or as desired. Other accessories can be added as desired. The doll's head is adorned with yarn “hair” and may also include a hat if desired. The entire assembly is attached by Velcro strips or spots so that it is easily removed as needed to replace the oxygen cylinder.

This decorative cover can help lessen the stigma of being tied to a piece of clinical medical equipment and can be tailored to the individual's interests. It can be changed as needed or desired.

In certain embodiments, the invention is a female doll. This can also be made as a male doll form, adding wings to make it appear as a fairy, making non-human forms such as a vase that can have removable silk flowers, a rocket ship, a tree, of animal forms such as a bear, giraffe, dog or cat. These can be tailored to the individual's interests.

The invention provides a profoundly positive affect on the mood of the patient. Patients are more likely to be willing to go out in public with the oxygen tank when properly adorned. The invention also provides a positive impact on people in the community who are drawn to the “doll” and engage with the patient in conversation and by taking an interest in her well-being. She can change the outfit as needed which keeps it interesting.

In certain embodiments of the invention, a cover for an oxygen tank, oxygen cylinder and/or cart is provided. In certain embodiments, the cover is a cloth “coat” that wraps around the tank and cart that has a doll-type head and arms attached.

In certain embodiments, the doll head covers the cylinder controls which are easily accessed if needed.

In certain embodiments, a dress is added over the doll form and can be changed seasonally or as desired. In certain embodiments, other accessories can be added as desired. In certain embodiments, the doll's head is adorned with yarn “hair” and may also include a hat if desired.

In certain embodiments, the entire assembly is attached by Velcro® strips or spots so that it is easily removed as needed to replace the oxygen cylinder.

In certain embodiments, this decorative cover can help lessen the stigma of being tied to a piece of clinical medical equipment and can be tailored to the individual's interests. It can be changed as needed or desired.

In certain embodiments, the prototype is a female doll. This can also be made as a male doll form.

In certain embodiments, the cover includes adding wings to make it appear as a fairy, making non-human forms such as a vase that can have removable silk flowers, a rocket ship, a tree, of animal forms such as a bear, giraffe, dog or cat.

In certain embodiments, the cover is tailored to the patient's interest.

Typically, the cover for the oxygen tank has a profoundly positive affect on the mood of the patient. When using the cover, patients are more likely to be willing to go out in public with her oxygen tank when properly adorned.

There is a positive impact on people in the community who are drawn to the “doll” and engage with the patient in conversation and by taking an interest in the patient's well-being.

In certain embodiments, the patient can change the outfit of the doll as needed which keeps it interesting.

In certain embodiments, the cover is insulative and keeps the oxygen tank protected.

In certain embodiments, the cover does not interference with the function of the tank.

In certain embodiments, the cover has a slot on its side and various openings, so that a user can adjust the flow of oxygen from the tank.

In certain embodiments, the cover has an opening to allow for the oxygen tank vales to be accessed.

In certain embodiments, the cover has multiple pieces and elements such that the cover has multiple pieces and such that the cover has multiple pieces that can cover the oxygen tank, while allowing for access to the oxygen tank.

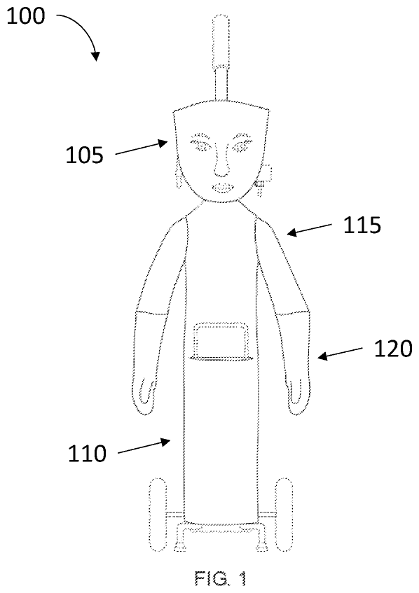

As depicted in FIG. 1, an oxygen cover of an embodiment of the present invention (100) is shown. In this particular embodiment, the cover is configured in appearance as a female doll having a face (105), dress (110), shirt (115) and hands (120). The inventive cover embodiment of the doll is shown covering and encompassing an oxygen tank (Not Shown, that is, the oxygen tank is “conceded” or “covered”).

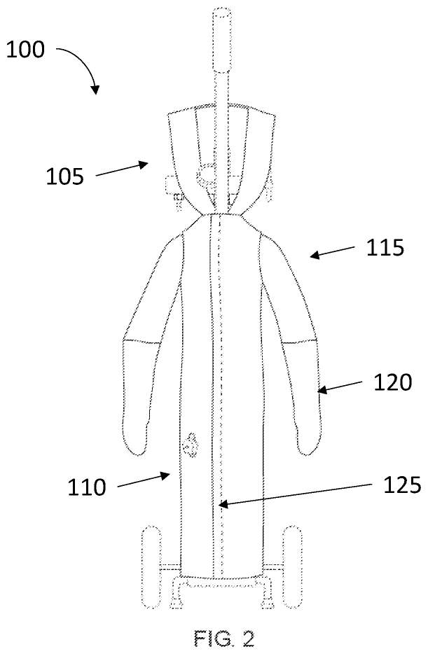

As depicted in FIG. 2, a Velcro® type fastener (125) may be used to affix the cover (100) to an oxygen tank.

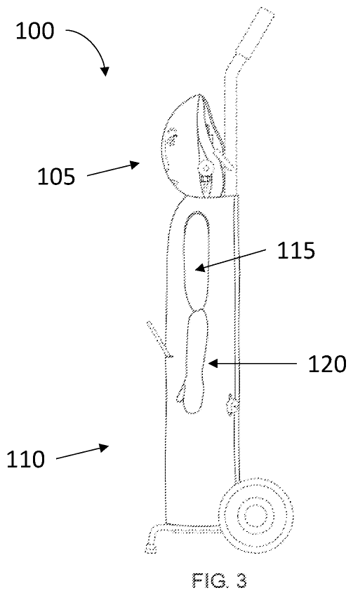

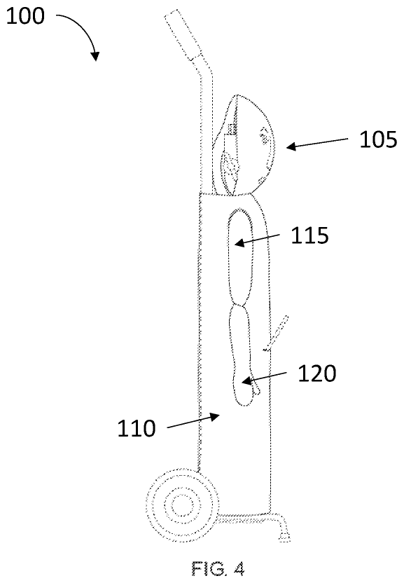

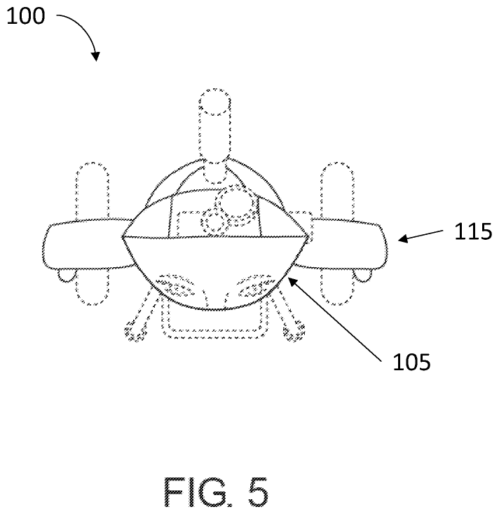

FIGS. 3-5 show additional various views of the inventive embodiment.

While the invention has been specifically described in connection with certain specific embodiments thereof, it is to be understood that this is by way of illustration and not of limitation and that various changes and modifications in form and details may be made thereto, and the scope of the appended claims should be construed as broadly as the prior art will permit.

The description of the invention is merely exemplary in nature, and thus, variations that do not depart from the gist of the invention are intended to be within the scope of the invention. Such variations are not to be regarded as a departure from the spirit and scope of the invention.

What is claimed is:

1. A cover system for a pressurized medical gas cylinder, the cover system comprising:
at least in part a flexible material;
wherein the cover system configuration is easily removable from and/or added to the pressurized medical gas cylinder,
wherein the cover system provides a less unsightly perception to and by a patient and others than that of the pressurized medical gas cylinder itself, and
wherein the cover system facilitates the operation and replacement of the pressurized medical gas cylinder.

2. The system of claim 1, wherein the cover further comprises at least one fastener, the at least one fastener configured to connect to the flexible material to the pressurized medical gas cylinder.

3. The system of claim 1, wherein the pressurized medical gas cylinder includes an associated and accompanying holding and travel cart, valves, tubing, dosing systems and masks, and wiring.

4. The system of claim 1, wherein the flexible material wraps around the pressurized medical gas cylinder, thereby providing a less unsightly perception to and by a patient and others than that of the pressurized medical gas cylinder itself.

5. The system of claim 1, wherein the flexible material is configured to include toys, dolls, cartoon characters, action figures, animals, public figures, and combinations thereof, on the flexible material

6. The system of claim 1, wherein the cover provides support for and to the pressurized medical gas cylinder.

7. The system of claim 1, wherein the cover protects the pressurized medical gas cylinder from the group consisting of rain, water, heat, flame, snow, cold, high barometric pressure, low barometric pressure, and combinations thereof.

8. The system of claim 1, wherein the cover includes a natural material and/or a synthetic material

9. The system of claim 1, wherein the cover is at least in part ridged.

10. A method to cover a pressurized medical gas cylinder, the method comprising the steps of:
providing a pressurized medical gas cylinder cover comprising at least in part a flexible material;
wherein, the cover is configured to be easily removed from and/or added to the pressurized medical gas cylinder;
wherein the cover system provides a less unsightly perception to and by a patient and others than that of the pressurized medical gas cylinder itself, and
wherein the cover system facilitates the operation and replacement of the pressurized medical gas cylinder.

11. The method of claim 10, wherein the provided cover further comprises at least one fastener.

12. The method of claim 10, wherein the pressurized medical gas cylinder includes an associated and accompanying holding and travel cart, valves, tubing, dosing systems and masks, and wiring.

13. The method of claim 10, wherein the pressurized medical gas cylinder includes associated and accompanying valves, tubing, dosing systems and masks, and wiring.

14. The method of claim 10, wherein the provided cover wraps around the pressurized medical gas cylinder.

15. The method of claim 10, wherein the provided cover is configured in appearance from the group consisting of toys, dolls, cartoon characters, action figures, animals, public figures, and combinations thereof.

16. The method of claim 10, wherein the provided cover provides support for and to the pressurized medical gas cylinder.

17. The method of claim 10, wherein the provided cover protects the pressurized medical gas cylinder from the group consisting of rain, water, heat, flame, snow, cold, high barometric pressure, low barometric pressure, and combinations thereof.

18. The method of claim 10, wherein the provided cover comprises at least in part a natural material and/or a synthetic material.

19. The system of claim 1, wherein the provided cover is at least in part ridged.
